(19) United States
(12) Patent Application Publication
(10) Pub. No.: US 2024/0279342 A1
(43) Pub. Date: Aug. 22, 2024

(54) COMPOSITIONS AND METHODS FOR IMMUNOTHERAPY

(71) Applicant: SURFACE ONCOLOGY, INC., Cambridge, MA (US)

(72) Inventors: James MOHAN, Cambridge, MA (US); Marisella Panduro SICHEVA, Cambridge, MA (US)

(21) Appl. No.: 17/775,981

(22) PCT Filed: Nov. 13, 2020
(86) PCT No.: PCT/US2020/060524

§ 371 Date: May 11, 2022

Related U.S. Application Data
(60) Provisional application No. 62/936,176, filed on Nov. 15, 2019.

(51) Int. Cl. C07K 16/28; A61K 39/00; A61K 45/06; A61P 35/00

(52) U.S. Cl. CPC C07K 16/283; A61K 45/06; A61P 35/00; C07K 16/2803; A61K 2039/505; C07K 2317/52; C07K 2317/55; C07K 2317/74

(57) ABSTRACT

The invention provides compositions and methods for engaging, coupling, or binding CD16 and CD112R to preferentially activate NK cells for use in treating cancer.

This application claims the benefit of priority to U.S. Provisional Application No. 62/936,176, filed Nov. 15, 2019 which is incorporated by reference in its entirety.

### SEQUENCE LISTING

The instant application contains a Sequence Listing which has been submitted electronically in ASCII format and is hereby incorporated by reference in its entirety. Said ASCII copy, created on Nov. 8, 2022, is named LU67074US01_SL.txt and is 1,501,851 bytes in size.

### BACKGROUND

Both the innate and adaptive arms of the immune system utilize highly specialized immune cells to patrol the body, searching for signs of malignancy. Innate immunity provides the first line of defense and a rapid response using mechanisms such as barriers and destructive peptides that are non-specific and naturally present. Natural killer (NK) cells are a type of lymphocyte that is part of the innate immune system and can recognize and destroy virally infected and tumor cells using granzymes stored in their cytoplasm.

Adaptive immunity develops over time in response to antigen and provides lasting immunity. Cytotoxic lymphocytes (CTLs), also known as CD8+ T cells are part of the adaptive immune response as they recognize virus and tumor derived antigens presented by antigen presenting cells (APCs). CTLs are activated by interaction with an APC such as a dendritic cell or macrophage. The APC presents the tumor antigens in the context of MHC molecules to the T cell receptor (TCR) on the T cell surface. During this cognate interaction, the APC provides a costimulatory signal which leads to T cell activation, T cell proliferation, and reduction or elimination of cells expressing the antigen via cytotoxic mechanisms.

Administration of anti-CD112R immunotherapy provides an opportunity to increase, enhance and sustain immune responses. CD112R is an inhibitory receptor primarily expressed by T cells and NK cells and competes for CD112 binding with the activating receptor CD226. The interaction of CD112 with CD112R is of higher affinity than with CD226 and thereby effectively regulates CD226 mediated cell activation. Anti-CD112R antibodies that block the interaction with CD112 limit inhibitory signaling directly downstream of CD112R while simultaneously promoting greater immune cell activation by increasing CD226 interactions with CD112. In in vitro studies, anti-CD112R antibodies have been shown to increase the proliferation, activation and cytotoxicity of immune effector cells.

CD112R mRNA expression is detected in a number of cancer tissues and based on predictive analysis using TCGA (The Cancer Genome Atlas) dataset. Its expression is strongest in tumors that are enriched for T and NK cells. In addition to being expressed on myeloid cells, the expression of the CD112R ligand, CD112, is routinely elevated on tumor cells of different cellular origins. Given these circumstances, engagement of CD112R on tumor infiltrating immune cells has a strong potential to negatively regulate local immune responses within the tumor microenvironment.

Despite the successes of anti-CD112R immunotherapy, improved therapies for treating cancer as well as therapies for treating cancers that are PD-1/PD-L1 resistant are needed. Therapeutic treatment with agents that couple CD112R and CD16 provide an opportunity to down modulate the inhibitory signaling that occurs putatively when CD112R expressing immune cells engage CD112 on tumor cells and/or myeloid cells within the tumor microenvironment and has the potential to enhance, increase and sustain anti-tumor immune responses. Provided herein are compositions and methods for use in coupling, simultaneously binding, and/or engaging CD112R and CD16 to treat cancer.

### BRIEF DESCRIPTION OF THE FIGURES

FIG. 1A shows a schematic of a tumor microenvironment.

FIG. 1B shows that CD112R expression is increased on activated NK and T cells.

FIG. 2 shows that CD112R is upregulated on CT26 tumor infiltrating NK and CD8⁺ T cells.

FIG. 3A shows a schematic of clone 35 coupling CD112R and CD16 on NK cells.

FIG. 3B shows the results of an NK activation assay with anti-CD112R antibodies: clone 35 (IgG1/IgG4), clone 38 (IgG1/IgG4), and clone 44 (IgG1/IgG4).

FIG. 4A shows that CD112R overexpression abrogates T cell activation.

FIG. 4B shows that clone 35 enhances T cell activation as compared to isotype control.

FIG. 5A shows the results of an in vitro NK cell activity assay.

FIG. 5B shows tumor volume after administration of anti-CD112R antibody (clone 46) in mouse IgG1 (which is analogous to human IgG4) and mouse IgG2a (which is analogous to human IgG1).

FIG. 6A shows that anti-CD112R activity is NK and T cell dependent.

FIG. 6B shows immunological memory in mice treated with anti-CD112R upon re-challenge.

FIGS. 7A and 7B show clone 35 compared to clone 35.4 in a NK activation assay in two donors.

FIGS. 8A-8B show Granzyme B+ (FIG. 8A) and percent interferon-γ+ (FIG. 8B) levels after treatment with anti-CD112R (clone 46), anti-TIGIT, or a combination of anti-CD112R (clone 46) and anti-TIGIT.

FIG. 9 shows tumor volume in a CT26 model of cancer for an anti-CD112R (clone 46) antibody in mIgG1/hIgG4 format versus mIgG2a/hIgG1 format.

FIG. 10A-10B show results of an in vivo study of changes in tumor volume upon administration of clone 46 in a mIgG2a/hIgG1 format.

FIG. 11A-11C show the results of additional experiments demonstrating 4-1BB induction on NK cells co-cultured with K562 cells in the presence of 1 μg/mL Clone 35 or Clone 35-Fab, compared to a hIgG1 isotype control antibody or control Fab (n=5 donors) (FIG. 11A), 4-1BB induction on NK cells co-cultured with K562 cells in the presence of 1 μg/mL Clone 35, Clone 35-IgG4, or with an IgG1 mutant (Clone 35-N297A), compared with hIgG1 isotype control (n=5) (FIG. 11B), and the impact of 1 μg/mL Clone 35 on NK cell activation following co-culture with K562 cells in the presence of 2 μg/mL anti-CD16, anti-CD32, or mIgG1 control Fab molecules (n=5). (* P<0.05, ** P<0.01: paired t-test) (FIG. 11C).

### DETAILED DESCRIPTION OF EMBODIMENTS OF THE INVENTION

### I. Definitions

In this application, the use of “or” means “and/or” unless stated otherwise. In the context of a multiple dependent claim, the use of “or” refers back to more than one preceding independent or dependent claim in the alternative only. The terms “comprising.” “including,” and “having” can be used interchangeably herein.

The terms “CD112R,” “PVR Related Immunoglobulin Domain Containing,” “CD112 Receptor,” “Poliovirus Receptor-Related Immunoglobulin Domain-Containing Protein” “Poliovirus Receptor Related Immunoglobulin Domain Containing,” “Nectin-2 Receptor,” “C7orf15,” and “Transmembrane Protein PVRIG” are all used interchangeably and refer to a native, human CD112R, unless otherwise specifically indicated (e.g. mouse CD112R, cynomolgus CD112R, etc.). The term includes full-length, unprocessed CD112R as well as any form of CD112R that results from processing in the cell. The term encompasses naturally occurring variants of human CD112R, e.g., splice variants or allelic variants. External ID's for CD112R gene include Entrez Gene: 79037, Ensembl: ENSG00000213413, OMIM: 617012, and UniProtKB: Q6DKI7.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations optionally resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂: diabodies: linear antibodies: single-chain antibody molecules (e.g. scFv); and multispecific antibodies formed from antibody fragments.

The term “block,” in the context of an interaction between two or more molecules, is used herein to refer to inhibition or prevention of said interaction between the two or more molecules, wherein the inhibition or prevention of said interaction between the two or more molecules is complete or nearly complete under at least one condition. A “nearly complete” inhibition is a percent inhibition of about 70-99.9%, and a “complete” inhibition is 100%. For example, a molecule is said to “block” an interaction between two or more other molecules if it completely or nearly completely inhibits such interaction at certain concentrations in a dose dependent manner.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “tumor” is used herein to refer to a cell or cells that comprise a cancer. The term “tumor growth” is used herein to refer to proliferation or growth by a cell or cells that comprise a cancer that leads to a corresponding increase in the size or extent of the cancer.

“CD16” is also known in the art as FcγRIII and is often found on the surface of natural killer (NK) cells, neutrophils, monocytes, and macrophages.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

Administration “in combination with” one or more further therapeutic agents includes simultaneous (concurrent) and consecutive (sequential) administration in any order. “Simultaneous binding” (and iterations thereof) refers to a composition that is capable of binding to a target or targets at the same time at any one time point. Simultaneous binding does not require binding to a target or targets at the same time at every time point.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present (numbering in this paragraph is according to the EU numbering system, also called the EU index, as described in Kabat et al., Sequences of Proteins of Immunological Interest, 5th Ed. Public Health Service, National Institutes of Health, Bethesda, M D, 1991).

“Framework,” “framework region,” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See. e.g., Portolano et al., J. Immunol. 150:880-887 (1993); Clarkson et al., Nature 352:624-628 (1991).

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the VL, the subgroup is subgroup kappa I as in Kabat et al., supra. In some embodiments, for the VH, the subgroup is subgroup III as in Kabat et al., supra.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3).

In some embodiments, an antibody is provided according to the Table of Sequences, wherein the isotype is human IgG1. In some embodiments, an antibody is provided according to the Table of Sequences, wherein the isotype is human IgG4. In some embodiments, an antibody is provided according to the Table of Sequences, wherein the isotype is human IgG4, wherein there is a single mutation at serine 228 to proline (S228P). In some embodiments, an antibody is provided according to the Table of Sequences, wherein the isotype is human IgG4, wherein there are two mutations at serine 228 to proline (S228P) and leucine 235 to glutamate (L235E). Sequences for the human IgG1 and IgG4 are shown in the Sequence Table at SEQ ID Nos: 40000 and 40001, respectively. Sequences for human IgG4 with one or two mutations are shown in 40002 and 40003, respectively. Throughout, where an antibody or clone number is provided, the antibody is in the IgG1 format. If the antibody or clone number is appended with a “0.4”, for example. “Clone 35.4”, the antibody is an IgG4 antibody having a constant region comprising SEQ ID NO: 40002. The S228P mutation occurs at position 228 in the literature. The S→P mutation occurs in clone 35.4 may be at position 229 but is still referred to herein as S228P. In general, all exemplified antibodies described herein comprise the human kappa light chain.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848: 79-87 (2007).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation or composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

Compositions for use in methods of simultaneously engaging, coupling, or binding CD16 and CD112R are provided. In some embodiments, methods for treating cancer are encompassed comprising administering one or more compositions that are capable of simultaneously coupling, engaging, and/or binding to CD112R and CD16. In some embodiments, the composition comprises one agent that is capable of simultaneous binding. In some embodiments, the composition comprises more than one agent that simultaneously binds by virtue of its simultaneous or near simultaneous administration.

In some embodiments, the composition is a multispecific antibody that binds to CD112R and CD16. In some embodiments, the composition comprises two agents, wherein one agent engages, couples, or binds CD112R and the other agent engages, couples, or binds CD16.

In some embodiments, compositions for use in a method of
a) treating cancer by preferentially activating NK cells; and/or
b) enhancing NK cell activation; and/or
c) enhancing NK cell activation and not enhancing T cell activation, are provided, comprising administering a composition that engages, couples, or binds CD16 and CD112R.

In some embodiments, the composition is a multispecific antibody, wherein the antibody binds to, blocks and/or activates CD16 and CD112R.

In some embodiments, the composition comprises a CD16 agonist and an agent that binds to and/or activates CD112R.

In some embodiments, the composition comprises an anti-CD16 antibody.

In some embodiments, the composition comprises an anti-CD112R antibody.

In some embodiments, the composition comprises an anti-CD16 antibody and an anti-CD112R antibody.

### 1. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for CD112R and the other is for CD16. In certain embodiments, one of the binding specificities is for CD112R, one is for CD16, and another is selected independently from or more of PD-1, PD-L1, CTLA-4, Lag-3, TIM-3, TIGIT, CD96, PVRL1, PVRL2, PVRL3, PVRL4, CD155, STING, CD47, CD39, and IL-27. Bispecific antibodies may also be used to localize cytotoxic agents to cells which express CD112R. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1): cross-linking two or more antibodies or fragments (see, e.g., U.S. Pat. No. 4,676,980, and Brennan et al., Science, 229: 81 (1985)): using leucine zippers to produce bi-specific antibodies (see, e.g., Kostelny et al., J. Immunol., 148(5): 1547-1553 (1992)): using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting Fantibody” or “DAF” comprising an antigen binding site that binds to CD112R as well as another, different antigen (see, US 2008/0069820, for example).

### 2. Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Natl Acad. Sci. USA 82:1499-1502 (1985): 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Natl Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg. M. S, and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12): 1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (e.g., U.S. Pat. No. 7,371,826).

See also Duncan & Winter, Nature 322:738-40 (1988): U.S. Pat. Nos. 5,648,260; 5,624,821; and WO 94/29351 concerning other examples of Fc region variants.

In some embodiments, an antibody is provided according to the Table of Sequences, wherein the isotype is human IgG1. In some embodiments, an antibody is provided according to the Table of Sequences, wherein the isotype is human IgG4. In some embodiments, an antibody is provided according to the Table of Sequences, wherein the isotype is human IgG4, wherein there is a single mutation at serine 228 to proline (S228P).

### 3. Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxy methylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Pharmaceutical Formulations

Pharmaceutical formulations of the described compositions are provided and may be used in the methods described herein. In some embodiments, the formulations are prepared by mixing the active ingredient so that it has the desired degree of purity with one or more optional pharmaceutically acceptable carriers, diluents, and/or excipients (Remington's Pharmaceutical Sciences 16th edition, Osol, A. Ed. (1980)), in the form of lyophilized formulations or aqueous solutions. Pharmaceutically acceptable carriers, diluents, and excipients are generally nontoxic to recipients at the dosages and concentrations employed, and include, but are not limited to: sterile water, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation or composition herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxy methylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations or compositions to be used for in vivo administration are generally sterile. Sterility may be readily accomplished, e.g., by filtration through sterile filtration membranes.

### C. Therapeutic Uses and Methods

Compositions for use in methods of simultaneously engaging, coupling, or binding CD16 and CD112R are provided. In some embodiments, methods for treating cancer are encompassed comprising administering one or more compositions that are capable of simultaneously coupling, engaging, and/or binding to CD112R and CD16. In some embodiments, the composition comprises one agent that is capable of simultaneous binding. In some embodiments, the composition comprises more than one agent that simultaneously binds by virtue of its simultaneous or near simultaneous administration.

In some embodiments, the composition is a multispecific antibody that binds to CD112R and CD16. In some embodiments, the composition comprises two agents, wherein one agent engages, couples, or binds CD112R and the other agent engages, couples, or binds CD16.

In some embodiments, compositions for use in a method of
a) treating cancer by preferentially activating NK cells; and/or
b) enhancing NK cell activation; and/or
c) enhancing NK cell activation and not enhancing T cell activation,

are provided, comprising administering a composition that engages, couples, or binds CD16 and CD112R.

In further aspects, the invention provides methods for treating diseases and/or disorders where blocking CD112R are desired. In some embodiments, methods for enhancing, increasing and/or sustaining an anti-tumor immune response in a subject having a tumor are provided comprising administering an agent or agent the couples, engages, or blocks CD16 and CD112R. In some embodiments, the tumor is cancerous. In some embodiments, methods for treating cancer in a subject having cancer are provided comprising administering an agent or agent the couples, engages, or blocks CD16 and CD112R.

The compositions described herein may be used, for example, for treating cancer. In some embodiments, methods for treating cancer are provided, comprising administering an effective amount of a CD16 and CD112R engaging, coupling, or binding composition or compositions.

Cancers can be cancers with solid tumors or blood malignancies (e.g., liquid tumors).

Non-limiting examples of cancers for treatment include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages. i.e., the myeloid cell line (which produces granulocytes, erythrocytes, thrombocytes, macrophages and mast cells) or lymphoid cell line (which produces B. T. NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (Ml), myeloblastic leukemia (M2: with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma: lymphomas, such as Hodgkin's lymphoma (HL), non-Hodgkin's lymphoma (NHL). B cell hematologic malignancy, e.g., B cell lymphomas, T cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T cell lymphoma/leukemia, mantle cell lymphoma, angio immunoblastic T cell lymphoma, angiocentric lymphoma, intestinal T cell lymphoma, primary mediastinal B-cell lymphoma, precursor T-lymphoblastic lymphoma, T-lymphoblastic; and lymphoma/leukaemia (T-Lbly/T-ALL), peripheral T cell lymphoma, lymphoblastic lymphoma, post-transplantation lymphoproliferative disorder, true histiocytic lymphoma, primary central nervous system lymphoma, primary effusion lymphoma. B cell lymphoma, lymphoblastic lymphoma (LBL), hematopoietic tumors of lymphoid lineage, acute lymphoblastic leukemia, diffuse large B-cell lymphoma. Burkitt's lymphoma, follicular lymphoma, diffuse histiocytic lymphoma (DHL), immunoblastic large cell lymphoma, precursor B-lymphoblastic lymphoma, cutaneous T cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T cell and B cell tumors, including but not limited to T cell disorders such as T-prolymphocytic leukemia (T-PLL), including of the small cell and cerebriform cell type: large granular lymphocyte leukemia (LGL) of the T cell type: a/d T-NHL hepatosplenic lymphoma: peripheral/post-thymic T cell lymphoma (pleomorphic and immunoblastic subtypes): angiocentric (nasal) T cell lymphoma: cancer of the head or neck, renal cancer, rectal cancer, cancer of the thyroid gland: acute myeloid lymphoma, as well as any combinations of said cancers. The methods described herein can also be used for treatment of metastatic cancers, unresectable, refractory cancers (e.g., cancers refractory to previous immunotherapy, e.g., with a blocking CTLA-4 or PD-1 antibody), and/or recurrent cancers.

In certain embodiments, a composition described herein is administered to subjects having a cancer that has exhibited an inadequate response to, or progressed on, a prior treatment, e.g., a prior treatment with an immuno-oncology or immunotherapy drug. In some embodiments, the cancer is refractory or resistant to a prior treatment, either intrinsically refractory or resistant (e.g., refractory to a PD-1 pathway antagonist), or a resistance or refractory state is acquired. For example, a composition described herein may be administered to subjects who are not responsive or not sufficiently responsive to a first therapy or who have disease progression following treatment, e.g., anti-PD-1 pathway antagonist treatment, either alone or in combination with another therapy (e.g., with an anti-PD-1 pathway antagonist therapy). In other embodiments, a composition described herein is administered to subjects who have not previously received (i.e., been treated with) an immuno-oncology agent, e.g., a PD-1 pathway antagonist.

### D. Combinations

Compositions of the invention can be used either alone or in combination with other agents in a therapy. For instance, a composition of the invention may be co-administered with at least one additional therapeutic agent (e.g., further comprising administering a second therapy).

In some embodiments, targeting an additional independent inhibitory pathway or combinations thereof has the potential to lead to further enhanced immune cell activation beyond monotherapy.

In some embodiments, the additional therapeutic agent or second agent is a chemotherapeutic agent, an opsonizing agent, a regulatory T cell (“Treg”) depleting agent, an antagonist of a target other than CD112R, or an agonist of a target other than CD112R. In certain embodiments, the second agent is a chemotherapeutic agent described herein or any known chemotherapeutic agent. In some embodiments, the second agent is an opsonizing agent, wherein the opsonizing agent is an antibody other than an anti-CD112R antibody that targets cancer or tumor cells. In some embodiments, the second agent is a Treg depleting agent described herein or any known Treg depleting agent. In some embodiments, the second agent is an antagonist of a target other than CD112R. In some embodiments, the second agent is an agonist of a target other than CD112R.

In some instances, the second agent targets an independent inhibitory pathway, such as, for example, a pathway involving PD-1, PD-L1, CTLA-4, Lag-3 or TIM-3. In some embodiments, the second agent antagonizes one or more of PD-1, PD-L1, CTLA-4, Lag-3 and TIM-3. Suitable antagonists for use in the combination therapy described herein, include, without limitation, ligands, antibodies (e.g., monoclonal antibodies and bispecific antibodies), and multivalent agents. In one embodiment, the antagonist is a fusion protein, e.g., an Fc fusion protein, such as AMP-244. In some embodiments, the PD-1 antagonist is an anti-PD-1 or anti-PD-L1 antibody.

An exemplary anti-PD-1 antibody is nivolumab (BMS-936558) or an antibody that comprises the CDRs or variable regions of one of antibodies 17D8, 2D3, 4H1, 5C4, 7D3, 5F4 and 4A11 described in WO 2006/121168. In certain embodiments, an anti-PD-1 antibody is MK-3475 (Lambrolizumab) described in WO2012/145493; AMP-514 described in WO 2012/145493; or PDR001. Further known PD-1 antibodies and other PD-1 inhibitors include those described in WO 2009/014708, WO 03/099196, WO 2009/114335, WO 2011/066389, WO 2011/161699, WO 2012/145493, U.S. Pat. Nos. 7,635,757 and 8,217,149, and U.S. Patent Publication No. 2009/0317368. Any of the anti-PD-1 antibodies disclosed in WO2013/173223 can also be used. An anti-PD-1 antibody that competes for binding with, and/or binds to the same epitope on PD-1 as, as one of these antibodies can also be used in combination treatments.

In some embodiments, the anti-PD-L1 antibody useful for the combination therapy is BMS-936559 (referred to as 12A4 in WO 2007/005874 and U.S. Pat. No. 7,943,743), or an antibody that comprises the CDRs or variable regions of 3G10, 12A4, 10A5, 5F8, 10H10, 1B12, 7H1, 11E6, 12B7 and 13G4, which are described in PCT Publication WO 07/005874 and U.S. Pat. No. 7,943,743. In certain embodiment an anti-PD-L1 antibody is MEDI4736 (also known as durvalumab and Anti-B7-H1), MPDL3280A (also known as atezolizumab and RG7446), MSB0010718C (also known as avelumab; WO2013/79174), or rHigM12B7. Any of the anti-PD-L1 antibodies disclosed in WO2013/173223, WO2011/066389, WO2012/145493, U.S. Pat. Nos. 7,635,757 and 8,217,149 and U.S. Publication No. 2009/145493 can also be used. Anti-PD-L1 antibodies that compete with and/or bind to the same epitope as that of any of these antibodies can also be used in combination treatments.

In certain embodiments, the composition of the disclosure can be used with a CTLA-4 antagonist, e.g., an anti-CTLA-4 antibody. In one embodiment, an anti-CTLA-4 antibody is an antibody selected from the group of: Yervoy R: (ipilimumab or antibody 10DI, described in PCT Publication WO 01/14424), tremelimumab (formerly ticilimumab, CP-675,206), monoclonal or an anti-CTLA-4 antibody described in any of the following publications: WO 98/42752: WO 00/37504: U.S. Pat. No. 6,207,156; Hurwitz et al. (1998) Pro. Natl. Acad. Sci. USA 95(17): 10067-10071: Camacho et al. (2004) J. Clin. Oncology 22(145): antibodiestract No. 2505 (antibody CP-675206); and Mokyr et al. (1998) Cancer Res. 58:5301-5304. Any of the anti-CTLA-4 antibodies disclosed in WO2013/173223 can also be used.

In some embodiments, a composition of the disclosure is used in combination with a LAG-3 (also referred to herein and by others as LAG3) antagonist. Examples of anti-LAG3 antibodies include antibodies comprising the CDRs or variable regions of antibodies 25F7, 26H10, 25E3, 8B7, 11F2 or 17E5, which are described in U.S. Patent Publication No. US2011/0150892, WO10/19570 and WO2014/008218. In one embodiment, an anti-LAG-3 antibody is BMS-986016. Other art recognized anti-LAG-3 antibodies that can be used include IMP731 and IMP-321, described in US 2011/007023, WO08/132601, and WO09/44273. Anti-LAG-3 antibodies that compete with and/or bind to the same epitope as that of any of these antibodies can also be used in combination treatments.

In some embodiments, targeting two or more of TIGIT, CD96 and CD112R receptors simultaneously increases CD226 mediated signaling beyond the anti-CD112R monotherapy. Therefore, in some embodiments, the second agent is an antagonist of TIGIT and/or CD96. Suitable antagonists for use in the combination therapy described herein, include, without limitation, ligands, antibodies (e.g., monoclonal antibodies and bispecific antibodies), and multivalent agents.

In some embodiments, members of the PVR gene family are upregulated on tumor cells and can exhibit intrinsic tumor-promoting properties. Targeting additional members of the PVR gene family in combination with anti-CD112R antibodies leads to enhanced sensitivity to tumors beyond monotherapy. Therefore, in some embodiments, the second agent is selected from one or more of an antagonist of PVRL1, PVRL2, PVRL3, PVRL4, and CD155. Suitable antagonists for use in the combination therapy described herein, include, without limitation, ligands, antibodies (e.g., monoclonal antibodies and bispecific antibodies), and multivalent agents.

STING agonists induce innate immune cell activation resulting in increased T cell priming and recruitment of immune cells into the tumor microenvironment. Targeting STING agonists in combination with CD112R has the potential to lead to an even further increase in T cell and NK cell recruitment and activation.

Increased anti-CD47 antibody mediated phagocytosis can lead to an increase in the presentation of cancer derived antigens by macrophages to T cells. Combination treatment with an anti-CD47 antibody and an anti-CD112R antibody, such as an anti-CD112R antibody provided herein provides an opportunity to enhance cancer antigen specific T cell responses and is fully encompassed herein.

Adenosine, via adenosine receptors expressed on immune cells, inhibits T cell and NK cell activation. Anti-CD39 antibodies inhibit the generation of adenosine by preventing hydrolysis of adenosine triphosphate (ATP). Combination treatment with an anti-CD39 antibody and an anti-CD112R antibody, such as an anti-CD112R antibody provided herein, provides an opportunity to further enhance CD112R therapy by inhibiting adenosine mediated cell signaling in immune cells.

Cytokines can effectively modulate T cell and NK cell activation. IL-27 is an immunosuppressive cytokine that inhibits T cell and NK cell mediated responses. Anti-IL-27 antibodies provide an opportunity to enhance CD112R therapy by limiting immunosuppressive cytokine signaling in immune cells. Thus, combination treatment with an anti-IL-27 antibody and an anti-CD112R antibody, such as an anti-CD112R antibody provided herein, is provided.

The compositions herein may also be provided before, substantially contemporaneous with, or after other modes of treatment, for example, surgery, chemotherapy, radiation therapy, or the administration of a biologic, such as another therapeutic antibody. In some embodiments, the cancer has recurred or progressed following a therapy selected from surgery, chemotherapy, and radiation therapy, or a combination thereof. For example, a CD112R antibody as described herein could be administered as adjunctive therapy when there is a risk that micrometastases can be present and/or in order to reduce the risk of a relapse.

For treatment of cancer, the combinations may be administered in conjunction with one or more additional anti-cancer agents, such as a chemotherapeutic agent, growth inhibitory agent, anti-cancer vaccine such as a gene therapy vaccine, anti-angiogenesis agent and/or anti-neoplastic composition.

In some embodiments, an anti-inflammatory drug may be administered with the combination, such as a steroid or a non-steroidal anti-inflammatory drug (NSAID). In cases where it is desirable to render aberrantly proliferative cells quiescent in conjunction with or prior to treatment with CD112R antibodies described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone. Aminoglutethimide, Estramustine, Medroxyprogesteroneacetate, Leuprolide, Flutamide, Toremifene, ZOLADEX®, can also be administered to the subject. When employing the methods or compositions described herein, other agents used in the modulation of tumor growth or metastasis in a clinical setting, such as antimimetics, can also be administered as desired.

Such combination therapies noted above encompass combined administration (where two or more therapeutic agents are included in the same or separate formulations or compositions), and separate administration, in which case, administration of the antibody of the invention can occur prior to, simultaneously, and/or following, administration of the additional therapeutic agent or agents. In some embodiments, administration of the anti-CD112R antibody and administration of an additional therapeutic agent occur within about one month, or within about one, two or three weeks, or within about one, two, three, four, five, or six days, of each other.

A composition of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Compositions of the invention can be formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. As used herein, a “split dose” is the division of single unit dose or total daily dose into two or more doses, e.g., two or more administrations of the single unit dose. The composition may be administered as “split dose.”

The composition need not be but is optionally formulated with one or more agents currently used to prevent or treat the disorder in question. The effective amount of such other agents depends on the amount of composition present in the formulation or composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate. In some embodiments, the composition is provided in a formulation for immediate release and the other agent is formulated for extended release or vice versa.

### E. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition may be an antibody. The label or package insert indicates that the composition is used for treating the condition of choice. Moreover, the article of manufacture may comprise (a) a first container with a composition contained therein, wherein the composition comprises an antibody; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate.

### III. EXAMPLES

### Example 1

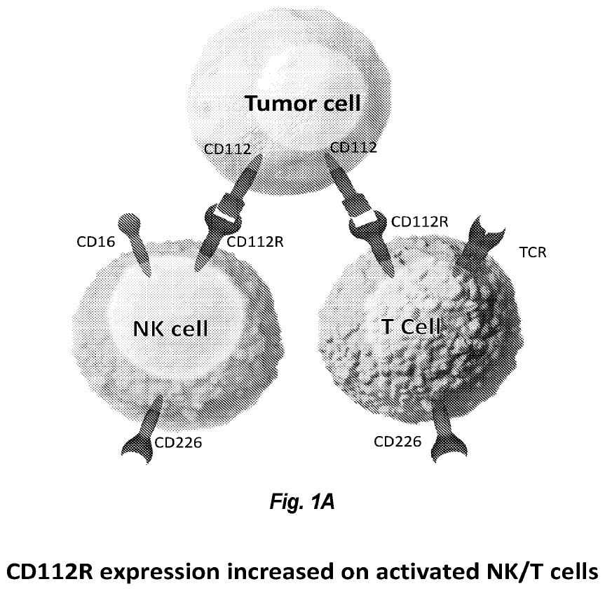

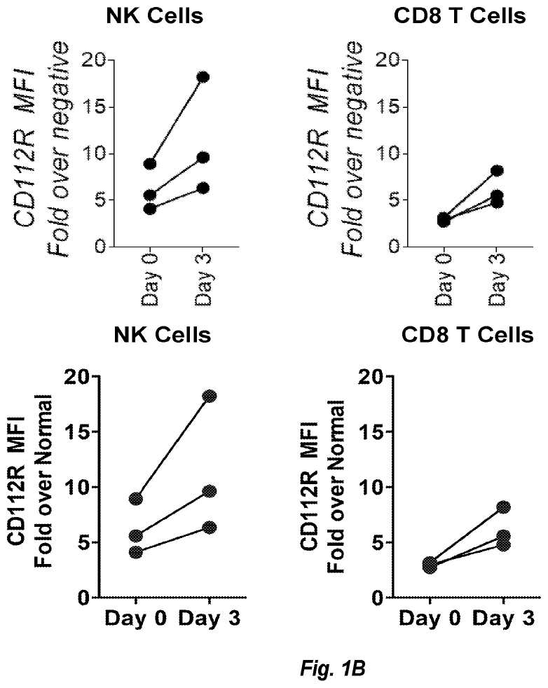

CD112R is an inhibitory receptor that is expressed on NK cells and T cells. CD112R suppresses immune cell activation through its association with the cell adhesion molecule CD112 (PVRL2), a ligand expressed on tumor cells that it competes for with the activating receptor CD226. CD112 binding to CD112R induces downstream signaling via an immunoreceptor tyrosine based inhibitory motif (ITIM) in the cytoplasmic tail, resulting in dampened effector cell activation. FIG. 1A provides a schematic, and FIG. 1B provides CD112R expression data for NK and CD8+ T cells.

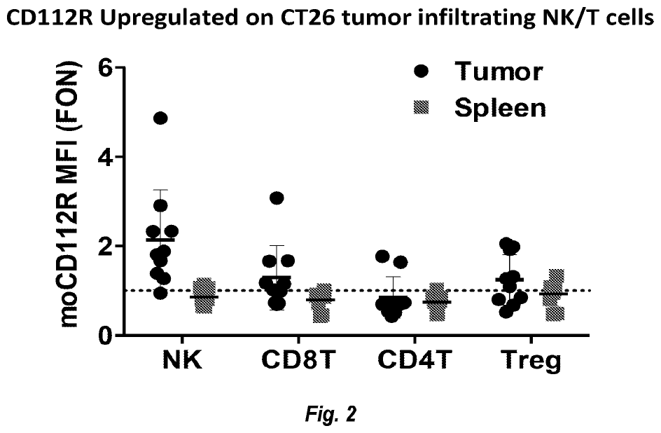

CD112R expression is upregulated on murine tumor infiltrating NK cells. See. FIG. 2. CD112R expression was evaluated on immune cell populations from the spleen and dissociated tumors in Balb/c mice implanted subcutaneously with CT-26 tumors by flow cytometry. CD112R expression is represented as fold over negative (isotype control).

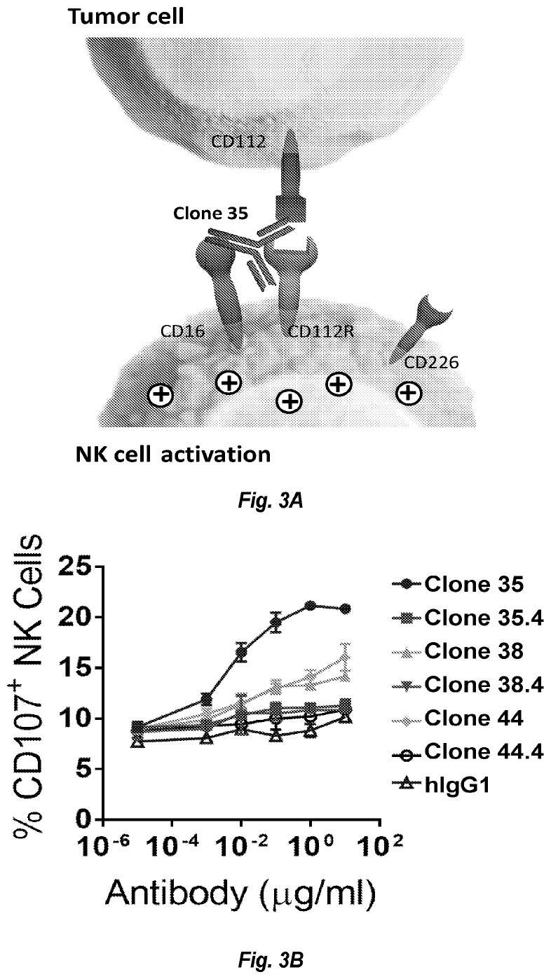

FIG. 3A shows a model for the therapeutic activity of clone 35 on NK cells. By engaging both CD16 and CD112R, clone 35 treatment promotes antitumor activity. FIG. 3B exemplifies that anti-CD112R antibodies with enhanced Fc effector function (hIgG1 Fc. Clone 35. Clone 38 and Clone 44) result in stronger NK cell degranulation in tumor cell co-cultures than Fc effector function low antibodies (hIgG4 Fc. Clone 35.4. Clone 38.4 and Clone 44.4). In particular. FIG. 3B shows enhanced NK cell mediated degranulation in response to tumor cells in the presence of enhanced Fc effector function CD112R antibodies as compared to Fc effector function low CD112R antibodies. Human NK cells and Raji CD112 cells were co-cultured for four hours with CD107a PE antibody in the presence of CD112R antibodies with IgG1 or IgG4.1 (S228P) isotypes. After co-culture. NK cell degranulation was determined by frequency of NK cells that were CD107a positive.

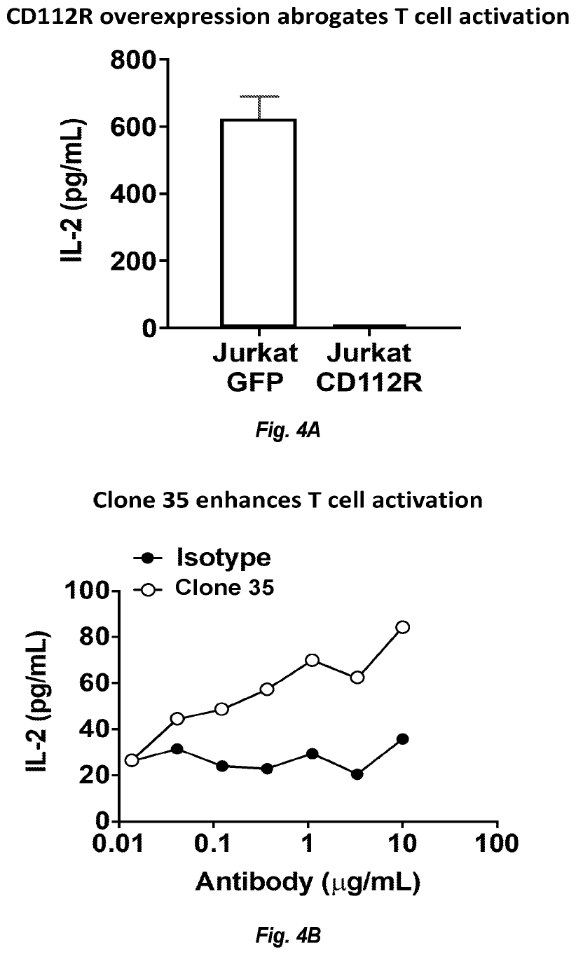

In FIG. 4A, overexpression of CD112R inhibits Jurkat cell TCR mediated activation. Jurkat cells transduced with either CD112R-ires-GFP (Jurkat CD112R) or GFP control vector (Jurkat GFP) were cocultured with TCR stimulator cells expressing membrane bound anti-CD3 scFv and CD112 ligand for 24 hrs. Activation was measured by IL-2 secretion into the supernatant by ELISA.

In FIG. 4B, clone 35 treatment increased TCR mediated activation of Jurkat. CD112R cells. Jurkat cells overexpressing CD112R were cocultured with TCR stimulator cells (Raji cells transduced with membrane bound anti-CD3 scFv and CD112 ligand) in the presence of clone 35 or isotype control antibody for 24 hrs. Activation of Jurkat cells was measured by IL-2 secretion into the supernatant by ELISA.

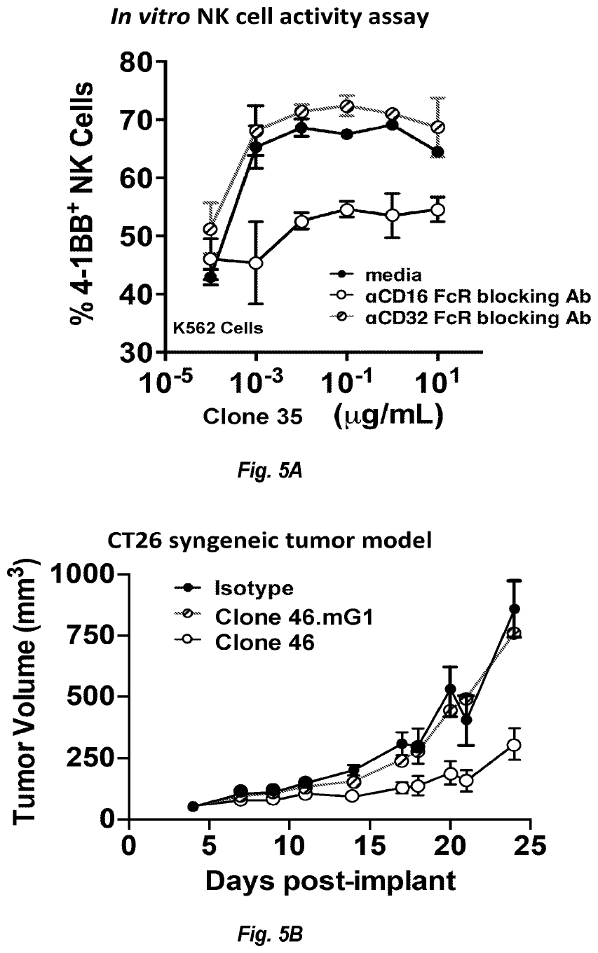

FIG. 5A shows that clone 35 mediated NK cell activation is partially abrogated by CD16 blockade. PBMCs from a single donor were cocultured with K562 target cells, clone 35 and F(ab′)2 antibodies that block either CD16 (Ancell, Clone 3G8), CD32 (Ancell, Clone 7.3) or media alone for 24 hr. NK cell activation was assessed by upregulation of 4-1BB expression by flow cytometry.

FIG. 5B shows stronger tumor growth inhibition in mice treated with an anti-CD112R antibody with enhanced Fc effector function (mouse IgG2a, Clone 46) compared to the same antibody engineered with low Fc-effector function (mouse IgG1, Clone 46.mG1). Graph is a summary of 3 experiments, N=44-45 per group.

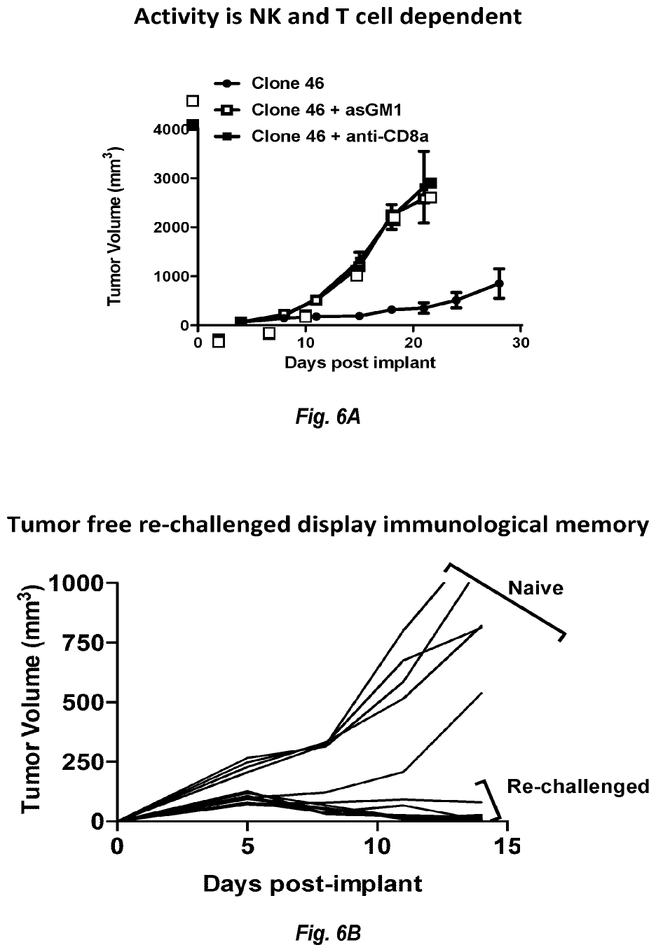

In vivo efficacy of CD112R blockade was evaluated in the CT26 syngeneic mouse tumor model following NK cell or CD8 T cell depletion. To deplete NK and CD8 T cells, mice were treated twice weekly for three weeks starting at randomization with Asialo-GM1 antibody (Biolegend: cat #146002: dose 100 uL/mouse: intraperitoneally) and anti-CD8a antibody (Bioxcell: cat #BE0085; 200 μg/mouse: intraperitoneally) respectively. BALB/cAnNTac female mice of 7 weeks of age (Taconic Biosciences, Catalog #BALB-F) were implanted subcutaneously in the right flank with 0.2×10⁶ CT26. WT (ATCC, Catalog #CRL-2638) in 0.1 mL 50% Geltrex (GIBCO, catalog #A1432-02) and 50% RPMI-1640 serum-free media (GIBCO, catalog #A10491-01). Mice with palpable tumors were randomized on day 4 post-implantation and treated intraperitoneally twice weekly for three weeks starting on the day of randomization with Clone 46 (anti-CD112R mouse IgG2a; 12.5 mg/kg: intraperitoneally). Tumor volumes were measured with a caliper every 2-3 days until tumors reached IACUC limit size (<2000 mm³). Tumor volume (mm³) was calculated as follows: width (mm)×[length (mm)]²×0.5. Results are presented in FIG. 6A. The graph depicts mean tumor volumes for each treatment group as a function of time. These results demonstrate that the therapeutic effect of anti-CD112R is significantly diminished following NK cell or CD8 T cell depletion.

Anti-tumor immunity was evaluated in anti-CD112R treated mice that exhibited complete responses from primary CT26. WT tumor challenges. For the primary challenge, BALB/cAnNTac female mice of 7 weeks of age (Taconic Biosciences, Catalog #BALB-F) were implanted subcutaneously in the right flank with 0.2×106 CT26. WT (ATCC, Catalog #CRL-2638) in 0.1 mL 50% Geltrex (GIBCO, catalog #A1432-02) and 50% RPMI-1640 serum-free media (GIBCO, catalog #A10491-01). Mice with palpable tumors were randomized on day 4 post-implantation and treated intraperitoneally twice weekly for three weeks starting on the day of randomization as follows in Table 1.

TABLE 1

| Group | Treatment | Dose (μg/mouse) |
|---|---|---|
| Isotype control | Mouse IgG2a | 500 |
| | isotype control | |
| Clone 46 | Anti-CD112R | 500 |
| | mouse IgG2a | |

Tumor volumes were measured with a caliper every 2-3 days until tumors reached IACUC limit size (<2000 mm3). Tumor volume (mm3) was calculated as follows: width (mm)×[length (mm)]2×0.5.

All Surviving mice at day 50 post implantation that lacked any discernable tumors were considered to be survivors/complete responders. Complete responder mice (n=8) from the anti-CD112R treated group were re-challenged via inoculation in the left flank with 1×10⁶ CT26. WT cells (ATCC, Catalog #CRL-2638) in 0.1 mL 50% Geltrex (GIBCO, catalog #A1432-02) and 50% RPMI-1640 serum-free media (GIBCO, catalog #A10491-01), a five-fold increase from the primary inoculation dose. As a control, age-matched naïve Balb/c female mice (n=5) were also similarly inoculated in the left flank with 1×106 CT26. WT cells in 0.1 mL 50% Geltrex and 50% RPMI-1640 serum-free media. Mice did not receive any further treatment. Tumor volumes were measured every 2-3 days until tumors reached IACUC limit size (<2000 mm3). Tumor volume (mm3) was calculated as follows: width (mm)×[length (mm)]2×0.5. Results are presented in FIG. 6B.

To assess the impact of CD112R antibodies on NK cell activation, clone 35 (hIgG1) and clone 35.4 (hIgG4) were evaluated in PBMC-tumor cell cocultures. Upregulation of CD137 (4-1BB), which has been previously established as a marker of NK cell activation (Baessler et al. (2010) Blood 115(15): André et al. (2018) Cell 175, 1731-1743) was measured on the NK cells from PBMCs cocultured with K562 target cells (chronic myelogenous leukemia cell line, ATCC #CCL-243) with anti-CD112R or isotype control antibodies.

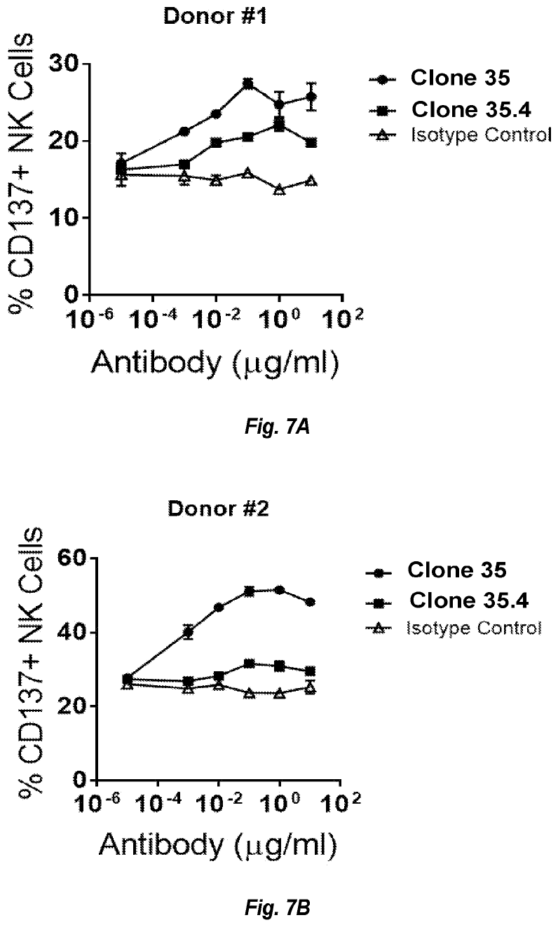

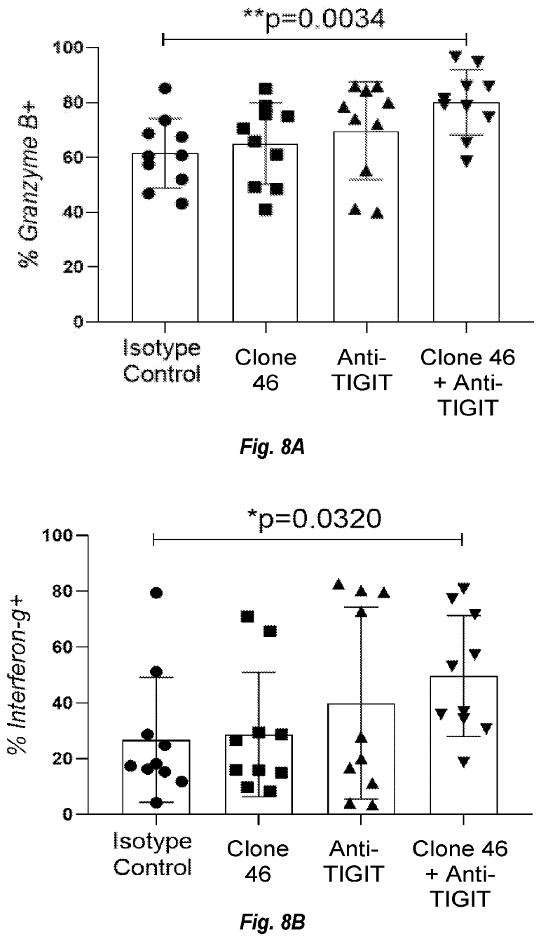

Briefly, frozen PBMCs isolated from the buffy coats of healthy donors were thawed, washed, resuspended in DMEM+10% FBS+1% Penicillin-Streptomycyin (D10) and plated into 96 well flat bottom plates at a concentration of 5×10⁵ cells per well and rested for 4 hours at 37° C. prior to adding target cells and antibodies. Next, in a first experiment (FIG. 7A) antibodies were diluted in D10 and added to each well at starting concentration of 10 μg/mL, with 10-fold serial dilutions. In the next experiment (FIGS. 8C-8D) a single concentration (1 μg/mL) of anti-CD112R or IgG1 isotype control antibody was added to each well. For both experiments, each condition was run in duplicate. K562 cells were then harvested, washed and resuspended in D10 and added to each well at a concentration of 5×10⁴ cells per well. The final volume for each well was 200 μl. The plates were then incubated for 16 hours at 37° C. After 16 hours, cells were then transferred to V bottom plates and washed twice in PBS+2% FBS. Cells were stained with Anti-CD3 FITC (Biolegend, #300306), Anti-NKp46 BV421 (Biolegend #331914) and anti-CD137 APC (Biolegend, #309810) in PBS+2% FBS for 30 minutes at 4° C. Cells were subsequently washed twice and resuspended in PBS+2% FBS. Data was acquired using a LSRFortessa X-20 (BD Biosciences) flow cytometer and analyzed with FlowJo software (Tree Star). NK cell activation was defined as the frequency of CD137+ cells within the CD3-NKp46+ lymphocyte gate.

Results from two individual donors from two independent experiments are presented in FIG. 7A-7B.

FIG. 8 demonstrates increased activation of intra-tumoral NK cells 72 hours post-single dose of anti-CD112R and anti-TIGIT combination therapy vis-à-vis isotype control. Activation was assessed as fraction of granzyme B+ (FIG. 8A) and interferon-γ+ (FIG. 8B)

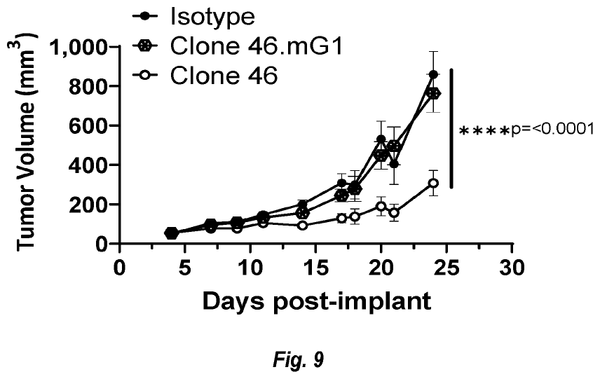

FIG. 9 reveals stronger tumor growth inhibition in mice treated with an anti-CD112R antibody with enhanced Fc effector function (mouse IgG2a, Clone 46) compared to the same antibody engineered with low Fc-effector function (mouse IgG1, Clone 46.mG1). Graph is a summary of 3 experiments, N=44-45 per group. Statistical analysis was performed by Mann-Whitney test on day 24 time-point.

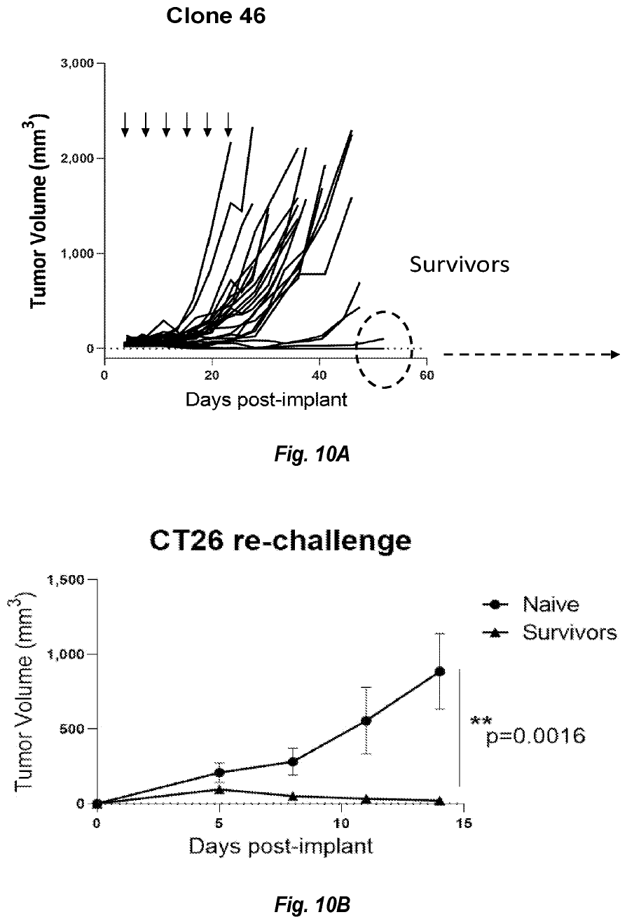

FIG. 10 shows a subset of mice rejected CT26 tumors after treatment with anti-CD112R.mG2a and exhibited no palpable tumors beyond day 50 of inoculation. These mice rapidly rejected of CT26 tumors upon re-challenge indicating that treatment with an enhanced Fc effector function CD112R antibody in tumor bearing mice lead to the development of immunological memory and protective immunity in a subset of mice.

### Example 2

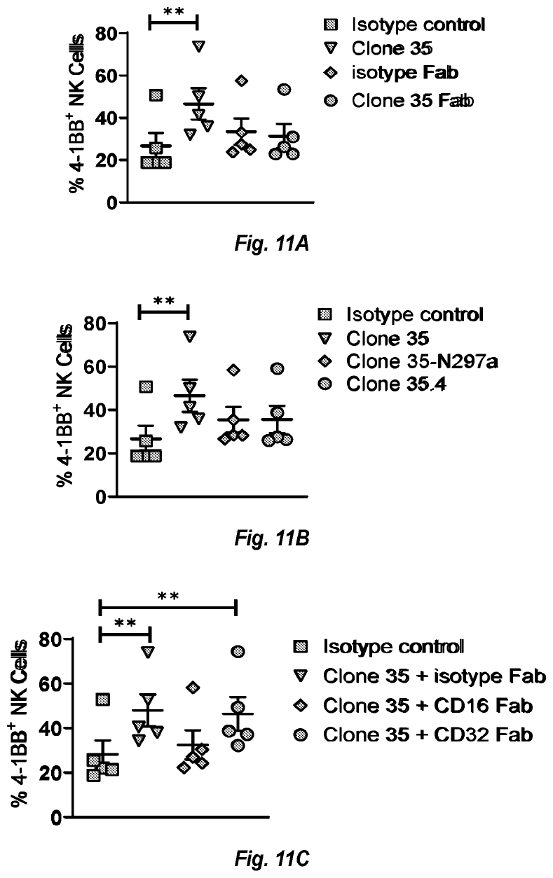

In a series of follow-up experiments, NK cell activation subsequent to co-culture with multiple permutations of anti-CD112R antibodies and anti-CD112R Fabs in PBMCs from additional donors was evaluated. The results demonstrate that enhanced clone 35 mediated NK cell activation requires both high Fc effector function and CD16 engagement in in vitro assays (FIGS. 11A-C).

FIG. 11A shows that clone 35 mediated NK cell activation is partially abrogated in the absence of an Fc backbone (antibody Fab). PBMCs from five donors were cocultured with K562 target cells with either full length clone 35, clone 35 Fab, full length isotype control or isotype control Fab for 24 hours. NK cell activation was assessed by upregulation of 4-1BB (CD137) expression by flow cytometry. Statistical analysis was performed by paired t test analysis.

FIG. 11B shows that clone 35 mediated NK cell activation is partially abrogated in absence of a glycosylated Fc backbone. Glycosylation of the Fc backbone at residue 297 significantly enhances the ability of IgG1 antibodies to bind to Fc receptors. The engineered mutation of the asparagine residue at position 297 to an alanine (N297A) prevents glycosylation of this residue and thus greatly diminishes the capacity of the antibody Fc backbone to engage Fc receptors (Wang et al, Protein Cell 2018). In this experiment PBMCs from five donors were cocultured with K562 target cells, clone 35, non-glycosylated clone 35 (Clone 35-N297A), effector function low clone 35 (hIgG4, Clone 35.4) or hIgG1 isotype control antibody for 24 hr. NK cell activation was assessed by upregulation of 4-1BB (CD137) expression by flow cytometry. Statistical analysis was performed by paired t test analysis.

FIG. 11C shows that clone 35 mediated NK cell activation is partially abrogated by CD16 blockade. PBMCs from five donors were cocultured with K562 target cells, clone 35 and Fab antibodies that block either CD16 (Ancell, Clone 3G8), CD32 (Ancell, Clone 7.3) or isotype control for 24 hr. NK cell activation was assessed by upregulation of 4-1BB (CD137) expression by flow cytometry. Statistical analysis was performed by paired t test analysis.

TABLE of
Sequences

| SEQ | Clone | | |
|---|---|---|---|
| ID NO | No | Description | Sequence |
| 1-200: | | | |
| Not used | | | |
| 201 | 44 | VH CDR1 | GTFDNYYIS |
| 202 | 44 | VH CDR2 | GIFPIFGTANYAQKFQG |
| 203 | 44 | VH CDR3 | AREVGHYSGSPYYMDV |
| 204 | 44 | VL CDR1 | RASQSINSWLA |
| 205 | 44 | VL CDR2 | DASSLES |
| 206 | 44 | VL CDR3 | QQVGPYLT |
| 207 | 44 | VH FR1 | QVQLVQSGAEVKKPGSSVKVSCKASG |
| 208 | 44 | VH FR2 | WVRQAPGQGLEWMG |
| 209 | 44 | VH FR3 | RVTITADESTSTAYMELSSLRSEDTAVYYC |
| 210 | 44 | VH FR4 | WGKGTTVTVSS |
| 211 | 44 | VH DNA | CAGGTGCAGCTGGTGCAGTCTGGGGCTGAGGTGAAGAAGCCTG |
| | | | GGTCCTCGGTGAAGGTCTCCTGCAAGGCTTCTGGAGGCACCTTC |
| | | | GACAACTATTACATCAGCTGGGTGCGACAGGCCCCTGGACAAGG |
| | | | GCTTGAGTGGATGGGAGGGATCTTCCCTATCTTCGGTACCGCAA |
| | | | ACTACGCACAGAAGTTCCAGGGCAGAGTCACGATTACCGCGGA |
| | | | CGAATCCACGAGCACAGCCTACATGGAGCTGAGCAGCCTGAGA |
| | | | TCTGAGGACACGGCGGTGTACTACTGCGCCAGAGAAGTCGGAC |
| | | | ACTACTCCGGCAGCCCATACTACATGGACGTATGGGGCAAGGGT |
| | | | ACAACTGTCACCGTCTCCTCA |
| 212 | 44 | VH Protein | QVQLVQSGAEVKKPGSSVKVSCKASGGTFDNYYISWVRQAPGQGL |
| | | | EWMGGIFPIFGTANYAQKFQGRVTITADESTSTAYMELSSLRSEDT |
| | | | AVYYCAREVGHYSGSPYYMDVWGKGTTVTVSS |
| 213 | 44 | VL FR1 | DIQMTQSPSTLSASVGDRVTITC |
| 214 | 44 | VL FR2 | WYQQKPGKAPKLLIS |
| 215 | 44 | VL FR3 | GVPSRFSGSGSGTEFTLTISSLQPDDFATYYC |
| 216 | 44 | VL FR4 | FGGGTKVEIK |
| 217 | 44 | VL DNA | GACATCCAGATGACCCAGTCTCCTTCCACCCTGTCTGCATCTGTA |
| | | | GGAGACAGAGTCACCATCACTTGCCGGGCCAGTCAGAGTATTAA |
| | | | TAGCTGGTTGGCCTGGTATCAGCAGAAACCAGGGAAAGCCCCTA |
| | | | AGCTCCTGATCTCCGATGCCTCCAGTTTGGAAAGTGGGGTCCCA |
| | | | TCAAGGTTCAGCGGCAGTGGATCTGGGACAGAATTCACTCTCAC |
| | | | CATCAGCAGCCTGCAGCCTGATGATTTTGCAACTTATTACTGCCA |
| | | | GCAGGTCGGCCCCTACCTCACTTTTGGCGGAGGGACCAAGGTTG |
| | | | AGATCAAA |
| 218 | 44 | VL Protein | DIQMTQSPSTLSASVGDRVTITCRASQSINSWLAWYQQKPGKAPKL |
| | | | LISDASSLESGVPSRFSGSGSGTEFTLTISSLQPDDFATYYCQQVGPY |
| | | | LTFGGGTKVEIK |
| 219-500: | | | |
| Not used | | | |
| 501 | 38 | VH CDR1 | FTFSGHLMS |
| 502 | 38 | VH CDR2 | AISGSAGETYYADSVKG |
| 503 | 38 | VH CDR3 | ARDAYYDDWSGWADWYFDL |
| 504 | 38 | VL CDR1 | RASQSVSRYLA |
| 505 | 38 | VL CDR2 | DASNRAT |
| 506 | 38 | VL CDR3 | QQVSLLPPT |
| 507 | 38 | VH FR1 | EVQLLESGGGLVQPGGSLRLSCAASG |
| 508 | 38 | VH FR2 | WVRQAPGKGLEWVS |
| 509 | 38 | VH FR3 | RFTISRDNSKNTLYLQMNSLRAEDTAVYYC |
| 510 | 38 | VH FR4 | WGRGTLVTVSS |
| 511 | 38 | VH DNA | GAGGTGCAGCTGTTGGAGTCTGGGGGAGGCTTGGTACAGCCTGG |
| | | | GGGGTCCCTGAGACTCTCCTGTGCAGCCTCTGGATTCACCTTTAG |
| | | | CGGACACCTAATGAGCTGGGTCCGCCAGGCTCCAGGGAAGGGG |
| | | | CTGGAGTGGGTCTCAGCTATTAGTGGATCCGCAGGTGAAACATA |
| | | | CTACGCAGACTCCGTGAAGGGCCGGTTCACCATCTCCAGAGACA |
| | | | ATTCCAAGAACACGCTGTATCTGCAAATGAACAGCCTGAGAGCC |
| | | | GAGGACACGGCGGTGTACTACTGCGCCAGAGATGCGTACTACG |
| | | | ACGACTGGAGCGGATGGGCCGATTGGTACTTCGATTTATGGGGG |
| | | | AGAGGTACCTTGGTCACCGTCTCCTCA |
| 512 | 38 | VH Protein | EVQLLESGGGLVQPGGSLRLSCAASGFTFSGHLMSWVRQAPGKGL |
| | | | EWVSAISGSAGETYYADSVKGRFTISRDNSKNTLYLQMNSLRAEDT |
| | | | AVYYCARDAYYDDWSGWADWYFDLWGRGTLVTVSS |
| 513 | 38 | VL FR1 | EIVLTQSPATLSLSPGERATLSC |
| 514 | 38 | VL FR2 | WYQQKPGQAPRLLIY |
| 515 | 38 | VL FR3 | GIPARFSGSGSGTDFTLTISSLEPEDFAVYYC |
| 516 | 38 | VL FR4 | FGGGTKVEIK |
| 517 | 38 | VL DNA | GAAATTGTGTTGACACAGTCTCCAGCCACCCTGTCTTTGTCTCCA |
| | | | GGGGAAAGAGCCACCCTCTCCTGCAGGGCCAGTCAGAGTGTTAG |
| | | | CAGGTACTTAGCCTGGTACCAACAGAAACCTGGCCAGGCTCCCA |
| | | | GGCTCCTCATCTATGATGCATCCAACAGGGCCACTGGCATCCCA |
| | | | GCCAGGTTCAGTGGCAGTGGGTCTGGGACAGACTTCACTCTCAC |
| | | | CATCAGCAGCCTAGAGCCTGAAGATTTTGCAGTTTATTACTGTC |
| | | | AGCAGGTCAGTCTCCTCCCTCCTACTTTTGGCGGAGGGACCAAG |
| | | | GTTGAGATCAAA |
| 518 | 38 | VL Protein | EIVLTQSPATLSLSPGERATLSCRASQSVSRYLAWYQQKPGQAPRLL |
| | | | IYDASNRATGIPARFSGSGSGTDFTLTISSLEPEDFAVYYCQQVSLLP |
| | | | PTFGGGTKVEIK |
| 519-700: | | | |
| Not used | | | |
| 701 | 35 | VH CDR1 | GTFSSAAIS |
| 702 | 35 | VH CDR2 | NIIPIVGIANYAQKFQG |
| 703 | 35 | VH CDR3 | ARDTGRGYTRHFWFDP |
| 704 | 35 | VL CDR1 | RASQSISSYLN |
| 705 | 35 | VL CDR2 | AASSLQS |
| 706 | 35 | VL CDR3 | QQSDILYT |
| 707 | 35 | VH FR1 | QVQLVQSGAEVKKPGSSVKVSCKASG |
| 708 | 35 | VH FR2 | WVRQAPGQGLEWMG |
| 709 | 35 | VH FR3 | RVTITADESTSTAYMELSSLRSEDTAVYYC |
| 710 | 35 | VH FR4 | WGQGTLVTVSS |
| 711 | 35 | VH DNA | CAGGTGCAGCTGGTGCAGTCTGGGGCTGAGGTGAAGAAGCCTG |
| | | | GGTCCTCGGTGAAGGTCTCCTGCAAGGCTTCTGGAGGCACCTTC |
| | | | AGCTCCGCCGCTATCAGCTGGGTGCGACAGGCCCCTGGACAAGG |
| | | | GCTTGAGTGGATGGGAAACATCATCCCTATCGTAGGTATAGCAA |
| | | | ACTACGCACAGAAGTTCCAGGGCAGAGTCACGATTACCGCGGA |
| | | | CGAATCCACGAGCACAGCCTACATGGAGCTGAGCAGCCTGAGA |
| | | | TCTGAGGACACGGCGGTGTACTACTGCGCCAGAGACACGGGAC |
| | | | GGGGATACACCAGACACTTCTGGTTTGACCCCTGGGGACAGGGT |
| | | | ACATTGGTCACCGTCTCCTCA |
| 712 | 35 | VH Protein | QVQLVQSGAEVKKPGSSVKVSCKASGGTFSSAAISWVRQAPGQGL |
| | | | EWMGNIIPIVGIANYAQKFQGRVTITADESTSTAYMELSSLRSEDTA |
| | | | VYYCARDTGRGYTRHFWFDPWGQGTLVTVSS |
| 713 | 35 | VL FR1 | DIQMTQSPSSLSASVGDRVTITC |
| 714 | 35 | VL FR2 | WYQQKPGKAPKLLIY |
| 715 | 35 | VL FR3 | GVPSRFSGSGSGTDFTLTISSLQPEDFATYYC |
| 716 | 35 | VL FR4 | FGGGTKVEIK |
| 717 | 35 | VL DNA | GACATCCAGATGACCCAGTCTCCATCCTCCCTGTCTGCATCTGTA |
| | | | GGAGACAGAGTCACCATCACTTGCCGGGCAAGTCAGAGCATTA |
| | | | GCAGCTATTTAAATTGGTATCAGCAGAAACCAGGGAAAGCCCCT |
| | | | AAGCTCCTGATCTATGCTGCATCCAGTTTGCAAAGTGGGGTCCC |
| | | | ATCAAGGTTCAGTGGCAGTGGATCTGGGACAGATTTCACTCTCA |
| | | | CCATCAGCAGTCTGCAACCTGAAGATTTTGCAACTTACTACTGTC |
| | | | AGCAAAGCGACATCCTCTACACTTTTGGCGGAGGGACCAAGGTT |
| | | | GAGATCAAA |
| 718 | 35 | VL Protein | DIQMTQSPSSLSASVGDRVTITCRASQSISSYLNWYQQKPGKAPKLL |
| | | | IYAASSLQSGVPSRFSGSGSGTDFTLTISSLQPEDFATYYCQQSDILY |
| | | | TFGGGTKVEIK |
| 719-90 | | | |
| not used | | | |
| 901 | 46 | VH CDR1 | FTFGDYAMS |
| 902 | 46 | VH CDR2 | FIGSKAYGGTTEYTASVKG |
| 903 | 46 | VH CDR3 | ARGPRRYTYGMDV |
| 904 | 46 | VL CDR1 | RASQSISSYLN |
| 905 | 46 | VL CDR2 | AASSLQS |
| 906 | 46 | VL CDR3 | QQSSTPLT |
| 907 | 46 | VH FR1 | EVQLVESGGGLVQPGRSLRLSCTASG |
| 908 | 46 | VH FR2 | WFRQAPGKGLEWVG |
| 909 | 46 | VH FR3 | RFTISRDGSKSIAYLQMNSLKTEDTAVYYC |
| 910 | 46 | VH FR4 | WGQGTTVTVSS |
| 911 | 46 | VH DNA | GAGGTGCAGCTGGTGGAGTCTGGGGGAGGCTTGGTACAGCCAG |
| | | | GGCGGTCCCTGAGACTCTCCTGTACAGCTTCTGGATTCACCTTTG |
| | | | GTGATTATGCTATGAGCTGGTTCCGCCAGGCTCCAGGGAAGGGG |
| | | | CTGGAGTGGGTAGGTTTCATTGGAAGCAAAGCTTATGGTGGGAC |
| | | | AACAGAATACACCGCGTCTGTGAAAGGCAGATTCACCATCTCAA |
| | | | GAGATGGTTCCAAAAGCATCGCCTATCTGCAAATGAACAGCCTG |
| | | | AAAACCGAGGACACGGCGGTGTACTACTGCGCCAGAGGACCAA |
| | | | GACGCTACACATACGGAATGGACGTATGGGGCCAGGGAACAAC |
| | | | TGTCACCGTCTCCTCA |
| 912 | 46 | VH Protein | EVQLVESGGGLVQPGRSLRLSCTASGFTFGDYAMSWFRQAPGKGL |
| | | | EWVGFIGSKAYGGTTEYTASVKGRFTISRDGSKSIAYLQMNSLKTE |
| | | | DTAVYYCARGPRRYTYGMDVWGQGTTVTVSS |
| 913 | 46 | VL FR1 | DIQMTQSPSSLSASVGDRVTITC |
| 914 | 46 | VL FR2 | WYQQKPGKAPKLLIY |
| 915 | 46 | VL FR3 | GVPSRFSGSGSGTDFTLTISSLQPEDFATYYC |
| 916 | 46 | VL FR4 | FGGGTKVEIK |
| 917 | 46 | VL DNA | GACATCCAGATGACCCAGTCTCCATCCTCCCTGTCTGCATCTGTA |
| | | | GGAGACAGAGTCACCATCACTTGCCGGGCAAGTCAGAGCATTA |
| | | | GCAGCTATTTAAATTGGTATCAGCAGAAACCAGGGAAAGCCCCT |
| | | | AAGCTCCTGATCTATGCTGCATCCAGTTTGCAAAGTGGGGTCCC |
| | | | ATCAAGGTTCAGTGGCAGTGGATCTGGGACAGATTTCACTCTCA |
| | | | CCATCAGCAGTCTGCAACCTGAAGATTTTGCAACTTACTACTGTC |
| | | | AGCAAAGCTCCACCCCCCTCACTTTTGGCGGAGGGACCAAGGTT |
| | | | GAGATCAAA |
| 918 | 46 | VL Protein | DIQMTQSPSSLSASVGDRVTITCRASQSISSYLNWYQQKPGKAPKLL |
| | | | IYAASSLQSGVPSRFSGSGSGTDFTLTISSLQPEDFATYYCQQSSTPL |
| | | | TFGGGTKVEIK |
| 919-39999 | | | |
| not used | | | |
| 40000 | | Human | ASTKGPSVFPLAPSSKSTSGGTAALGCLVKDYFPEPVTVSWNSGAL |
| | | IgG1 | TSGVHTFPAVLQSSGLYSLSSVVTVPSSSLGTQTYICNVNHKPSNTK |
| | | Constant | VDKKVEPKSCDKTHTCPPCPAPELLGGPSVFLFPPKPKDTLMISRTP |
| | | Region | EVTCVVVDVSHEDPEVKFNWYVDGVEVHNAKTKPREEQYNSTYR |
| | | | VVSVLTVLHQDWLNGKEYKCKVSNKALPAPIEKTISKAKGQPREP |
| | | | QVYTLPPSRDELTKNQVSLTCLVKGFYPSDIAVEWESNGQPENNYK |
| | | | TTPPVLDSDGSFFLYSKLTVDKSRWQQGNVFSCSVMHEALHNHYT |
| | | | QKSLSLSPGK |
| 40001 | | Human | ASTKGPSVFPLAPCSRSTSESTAALGCLVKDYFPEPVTVSWNSGALT |
| | | IgG4 | SGVHTFPAVLQSSGLYSLSSVVTVPSSSLGTKTYTCNVDHKPSNTK |
| | | Constant | VDKRVESKYGPPCPSCPAPEFLGGPSVFLFPPKPKDTLMISRTPEVTC |
| | | Region | VVVDVSQEDPEVQFNWYVDGVEVHNAKTKPREEQFNSTYRVVSV |
| | | (terminal K | LTVLHQDWLNGKEYKCKVSNKGLPSSIEKTISKAKGQPREPQVYTL |
| | | absent)* | PPSQEEMTKNQVSLTCLVKGFYPSDIAVEWESNGQPENNYKTTPPV |
| | | | LDSDGSFFLYSRLTVDKSRWQEGNVFSCSVMHEALHNHYTQKSLS |
| | | | LSLG- |
| | | | *The terminal K is cleaved during cell expression |
| | | | but is encoded for in the DNA sequence |
| 40002 | | Human | ASTKGPSVFPLAPCSRSTSESTAALGCLVKDYFPEPVTVSWNSGALT |
| | | IgG4 | SGVHTFPAVLQSSGLYSLSSVVTVPSSSLGTKTYTCNVDHKPSNTK |
| | | Constant | VDKRVESKYGPPCPPCPAPEFLGGPSVFLFPPKPKDTLMISRTPEVT |
| | | Region | CVVVDVSQEDPEVQFNWYVDGVEVHNAKTKPREEQFNSTYRVVS |
| | | (S228P; | VLTVLHQDWLNGKEYKCKVSNKGLPSSIEKTISKAKGQPREPQVYT |
| | | bolded) | LPPSQEEMTKNQVSLTCLVKGFYPSDIAVEWESNGQPENNYKTTPP |
| | | (terminal K | VLDSDGSFFLYSRLTVDKSRWQEGNVFSCSVMHEALHNHYTQKSL |
| | | absent)* | SLSLG- |
| | | | *The terminal K is cleaved during cell expression |
| | | | but is encoded for in the DNA sequence |
| 40003 | | Human | ASTKGPSVFPLAPCSRSTSESTAALGCLVKDYFPEPVTVSWNSGALT |
| | | IgG4 | SGVHTFPAVLQSSGLYSLSSVVTVPSSSLGTKTYTCNVDHKPSNTK |
| | | Constant | VDKRVESKYGPPCPPCPAPEFEGGPSVFLFPPKPKDTLMISRTPEVT |
| | | Region | CVVVDVSQEDPEVQFNWYVDGVEVHNAKTKPREEQFNSTYRVVS |
| | | (S228P) | VLTVLHQDWLNGKEYKCKVSNKGLPSSIEKTISKAKGQPREPQVYT |
| | | (L235E) | LPPSQEEMTKNQVSLTCLVKGFYPSDIAVEWESNGQPENNYKTTPP |
| | | (terminal K | VLDSDGSFFLYSRLTVDKSRWQEGNVFSCSVMHEALHNHYTQKSL |
| | | absent)* | SLSLG- |
| | | | *The terminal K is cleaved during cell expression |
| | | | but is encoded for in the DNA sequence |
| 40004 | 35 | Full length | CAAGTTCAGCTGGTGCAGAGCGGCGCTGAGGTGAAAAAGCCCG |
| | | clone 35 | GCAGCTCCGTGAAGGTGAGCTGCAAGGCCTCCGGCGGAACCTTC |
| | | heavy chain | TCCTCCGCTGCCATCTCTTGGGTGAGGCAAGCTCCCGGTCAAGG |
| | | DNA no | TTTAGAGTGGATGGGCAACATCATCCCCATCGTGGGCATCGCCA |
| | | leader | ACTACGCCCAGAAGTTCCAAGGTCGTGTGACCATCACCGCCGAC |
| | | | GAGTCCACCTCCACCGCCTACATGGAGCTGTCCTCTTTAAGGTCC |
| | | | GAGGACACCGCCGTGTACTACTGCGCTCGTGACACTGGTCGTGG |
| | | | ATACACTCGTCACTTCTGGTTCGACCCTTGGGGCCAAGGTACAC |
| | | | TGGTGACCGTGAGCTCCGCTAGCACCAAGGGCCCATCGGTCTTC |
| | | | CCCCTGGCACCCTCCTCCAAGAGCACCTCTGGGGGCACAGCGGC |
| | | | CCTGGGCTGCCTGGTCAAGGACTACTTCCCCGAACCGGTGACGG |
| | | | TGTCGTGGAACTCAGGCGCCCTGACCAGCGGCGTGCACACCTTC |
| | | | CCGGCCGTCCTACAGTCCTCAGGACTCTACTCCCTCAGCAGCGT |
| | | | GGTGACCGTGCCCTCCAGCAGCTTGGGCACCCAGACCTACATCT |
| | | | GCAACGTGAATCACAAGCCCAGCAACACCAAGGTGGACAAGAA |
| | | | GGTTGAGCCCAAATCTTGTGACAAAACTCACACATGCCCACCGT |
| | | | GCCCAGCACCTGAACTCCTGGGGGGACCGTCAGTCTTCCTCTTC |
| | | | CCCCCAAAACCCAAGGACACCCTCATGATCTCCCGGACCCCTGA |
| | | | GGTCACATGCGTGGTGGTGGACGTGAGCCACGAAGACCCTGAG |
| | | | GTCAAGTTCAACTGGTACGTGGACGGCGTGGAGGTGCATAATGC |
| | | | CAAGACAAAGCCGCGGGAGGAGCAGTACAACAGCACGTACCGT |
| | | | GTGGTCAGCGTCCTCACCGTCCTGCACCAGGACTGGCTGAATGG |
| | | | CAAGGAGTACAAGTGCAAGGTCTCCAACAAAGCCCTCCCAGCCC |
| | | | CCATCGAGAAAACCATCTCCAAAGCCAAAGGGCAGCCCCGAGA |
| | | | ACCACAGGTGTACACCCTGCCCCCATCCCGGGACGAGCTGACCA |
| | | | AGAACCAGGTCAGCCTGACCTGCCTGGTCAAAGGCTTCTATCCC |
| | | | AGCGACATCGCCGTGGAGTGGGAGAGCAATGGGCAGCCGGAGA |
| | | | ACAACTACAAGACCACGCCTCCCGTGCTGGACTCCGACGGCTCC |
| | | | TTCTTCCTCTACAGCAAGCTCACCGTGGACAAGAGCAGGTGGCA |
| | | | GCAGGGGAACGTCTTCTCATGCTCCGTGATGCATGAGGCTCTGC |
| | | | ACAACCACTACACGCAGAAGAGCCTCTCCCTGTCTCCGGGTAAA |
| | | | TGA |
| 40005 | 35 | Full length | GACATCCAGATGACCCAGTCCCCTTCCTCTTTATCCGCTTCCGTC |
| | | clone 35 | GGAGATCGTGTGACCATCACTTGTCGGGCCTCCCAGTCCATCAG |
| | | light chain | CTCCTATTTAAACTGGTACCAGCAGAAGCCCGGCAAGGCCCCCA |
| | | DNA no | AGCTGCTGATCTACGCCGCTTCTTCTTTACAGTCCGGCGTGCCTT |
| | | leader | CTCGTTTTTCTGGCTCCGGCTCTGGCACCGATTTCACTTTAACCA |
| | | | TCTCCTCCTTACAGCCCGAGGACTTCGCCACCTACTACTGCCAGC |
| | | | AGTCCGACATCCTGTACACCTTCGGCGGAGGCACCAAGGTGGAG |
| | | | ATCAAGCGTACGGTGGCTGCACCATCTGTCTTCATCTTCCCGCCA |
| | | | TCTGATGAGCAGTTGAAATCTGGAACTGCCTCTGTTGTGTGCCTG |
| | | | CTGAATAACTTCTATCCCAGAGAGGCCAAAGTACAGTGGAAGGT |
| | | | GGATAACGCCCTCCAATCGGGTAACTCCCAGGAGAGTGTCACAG |
| | | | AGCAGGACAGCAAGGACAGCACCTACAGCCTCAGCAGCACCCT |
| | | | GACGCTGAGCAAAGCAGACTACGAGAAACACAAAGTCTACGCC |
| | | | TGCGAAGTCACCCATCAGGGCCTGAGCTCGCCCGTCACAAAGAG |
| | | | CTTCAACAGGGGAGAGTGTTGA |
| 40006 | 35.4 | Full length | CAAGTTCAGCTGGTGCAGAGCGGCGCTGAGGTGAAAAAGCCCG |
| | | clone 35.4 | GCAGCTCCGTGAAGGTGAGCTGCAAGGCCTCCGGCGGAACCTTC |
| | | heavy chain | TCCTCCGCTGCCATCTCTTGGGTGAGGCAAGCTCCCGGTCAAGG |
| | | DNA no | TTTAGAGTGGATGGGCAACATCATCCCCATCGTGGGCATCGCCA |
| | | leader | ACTACGCCCAGAAGTTCCAAGGTCGTGTGACCATCACCGCCGAC |
| | | | GAGTCCACCTCCACCGCCTACATGGAGCTGTCCTCTTTAAGGTCC |
| | | | GAGGACACCGCCGTGTACTACTGCGCTCGTGACACTGGTCGTGG |
| | | | ATACACTCGTCACTTCTGGTTCGACCCTTGGGGCCAAGGTACAC |
| | | | TGGTGACCGTGAGCTCCGCTAGCACCAAGGGCCCATCCGTCTTC |
| | | | CCCCTGGCGCCCTGCTCCAGGAGCACCTCCGAGAGCACAGCCGC |
| | | | CCTGGGCTGCCTGGTCAAGGACTACTTCCCCGAACCGGTGACGG |
| | | | TGTCGTGGAACTCAGGCGCCCTGACCAGCGGCGTGCACACCTTC |
| | | | CCGGCTGTCCTACAGTCCTCAGGACTCTACTCCCTCAGCAGCGT |
| | | | GGTGACCGTGCCCTCCAGCAGCTTGGGCACGAAGACCTACACCT |
| | | | GCAACGTAGATCACAAGCCCAGCAACACCAAGGTGGACAAGAG |
| | | | AGTTGAGTCCAAATATGGTCCCCCATGCCCACCATGCCCAGCAC |
| | | | CTGAGTTCCTGGGGGGACCATCAGTCTTCCTGTTCCCCCCAAAA |
| | | | CCCAAGGACACTCTCATGATCTCCCGGACCCCTGAGGTCACGTG |
| | | | CGTGGTGGTGGACGTGAGCCAGGAAGACCCCGAGGTCCAGTTC |
| | | | AACTGGTACGTGGATGGCGTGGAGGTGCATAATGCCAAGACAA |
| | | | AGCCGCGGGAGGAGCAGTTCAACAGCACGTACCGTGTGGTCAG |
| | | | CGTCCTCACCGTCCTGCACCAGGACTGGCTGAACGGCAAGGAGT |
| | | | ACAAGTGCAAGGTCTCCAACAAAGGCCTCCCGTCCTCCATCGAG |
| | | | AAAACCATCTCCAAAGCCAAAGGGCAGCCCCGAGAGCCACAGG |
| | | | TGTACACCCTGCCCCCATCCCAGGAGGAGATGACCAAGAACCAG |
| | | | GTCAGCCTGACCTGCCTGGTCAAAGGCTTCTACCCCAGCGACAT |
| | | | CGCCGTGGAGTGGGAGAGCAATGGGCAGCCGGAGAACAACTAC |
| | | | AAGACCACGCCTCCCGTGCTGGACTCCGACGGCTCCTTCTTCCTC |
| | | | TACAGCAGGCTAACCGTGGACAAGAGCAGGTGGCAGGAGGGGA |
| | | | ATGTCTTCTCATGCTCCGTGATGCATGAGGCTCTGCACAACCACT |
| | | | ACACACAGAAGAGCCTCTCCCTGTCTCTGGGTAAATGA |
| 40007 | 35.4 | Full length | GACATCCAGATGACCCAGTCCCCTTCCTCTTTATCCGCTTCCGTC |
| | | clone 35.4 | GGAGATCGTGTGACCATCACTTGTCGGGCCTCCCAGTCCATCAG |
| | | light chain | CTCCTATTTAAACTGGTACCAGCAGAAGCCCGGCAAGGCCCCCA |
| | | DNA no | AGCTGCTGATCTACGCCGCTTCTTCTTTACAGTCCGGCGTGCCTT |
| | | leader | CTCGTTTTTCTGGCTCCGGCTCTGGCACCGATTTCACTTTAACCA |
| | | | TCTCCTCCTTACAGCCCGAGGACTTCGCCACCTACTACTGCCAGC |
| | | | AGTCCGACATCCTGTACACCTTCGGCGGAGGCACCAAGGTGGAG |
| | | | ATCAAGCGTACGGTGGCTGCACCATCTGTCTTCATCTTCCCGCCA |
| | | | TCTGATGAGCAGTTGAAATCTGGAACTGCCTCTGTTGTGTGCCTG |
| | | | CTGAATAACTTCTATCCCAGAGAGGCCAAAGTACAGTGGAAGGT |
| | | | GGATAACGCCCTCCAATCGGGTAACTCCCAGGAGAGTGTCACAG |
| | | | AGCAGGACAGCAAGGACAGCACCTACAGCCTCAGCAGCACCCT |
| | | | GACGCTGAGCAAAGCAGACTACGAGAAACACAAAGTCTACGCC |
| | | | TGCGAAGTCACCCATCAGGGCCTGAGCTCGCCCGTCACAAAGAG |
| | | | CTTCAACAGGGGAGAGTGTTGA |

1. A method of
i) treating cancer by preferentially activating NK cells; and/or
ii) enhancing NK cell activation; and/or
iii) enhancing NK cell activation and not enhancing T cell activation, comprising administering to a human in need thereof a composition that engages, couples, or binds CD16 and CD112R.

2. The method of claim 1, wherein the composition is a multispecific antibody, wherein the antibody binds to, blocks, and/or activates CD16 and CD112R.

3. The method of claim 1, wherein the composition comprises a CD16 agonist and an agent that binds to and/or activates CD112R.

4. The method of claim 1, wherein the composition comprises an anti-CD16 antibody.

5. The method of claim 1, wherein the composition comprises an anti-CD112R antibody.

6. The method of claim 1, wherein the composition comprises an anti-CD16 antibody and an anti-CD112R antibody.

7. The method of claim 1, wherein the composition comprises an anti-CD112R antibody and the antibody is an IgG1.

8. The method of claim 1, wherein the cancer is carcinoma, lymphoma, blastoma, sarcoma, or leukemia.

9. The method of claim 8, wherein the cancer is squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, or head and neck cancer.

10. The method of claim 1, further comprising administering a second therapy.

11. The method of claim 10, wherein the second therapy is radiotherapy or surgery.

12. The method of claim 10, wherein the second therapy is administration of a chemotherapy, an opsonizing agent, or a regulatory T cell depleting agent.

13. The method of claim 10, wherein the second therapy is administration of an antagonist of PD-1, PD-L1, CTLA-4, Lag-3 or TIM-3.

14. The method of claim 10, wherein the second therapy is administration of an antagonist of TIGIT or CD96.

15. The method of claim 10, wherein the second therapy is administration of an antagonist of PVRL1, PVRL2, PVRL3, PVRL4, and or CD155.

16. The method of claim 10, wherein the second therapy is administration of an antagonist of CD47.

17. The method of claim 10, wherein the second therapy is administration of an antagonist of CD39.

18. The method of claim 10, wherein the second therapy is administration of an antagonist of IL-27.

19. The method of claim 10, wherein the second therapy is administration of a STING agonist.

20. The method of claim 13, wherein the antagonist is an antibody.
